(19) United States
(12) Patent Application Publication
(10) Pub. No.: US 2010/0151114 A1
(43) Pub. Date: Jun. 17, 2010

(54) IN-LINE TREATMENT OF YARN PRIOR TO CREATING A FABRIC

(75) Inventor: Russell M. Parrott, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/640,655

(22) Filed: Dec. 17, 2009

Related U.S. Application Data
(60) Provisional application No. 61/138,374, filed on Dec. 17, 2008.

(51) Int. Cl. B05D 3/10; A61F 2/08

(52) U.S. Cl. 427/2.26; 623/14.12

(57) ABSTRACT

A woven orthopedic implant for cartilage replacement having layered functionality and a method of forming the same. The woven orthopedic implant may include bottom layer of fibers that promotes anchoring to bone, and intermediate layer of fibers that promotes soft tissue attachment, and a top layer of fibers that promotes lubrication. The method may involve treating the surfaces of fibers before weaving the fibers together.

### CROSS-REFERENCE TO RELATED APPLICATION

This application claims priority from U.S. Provisional Patent Application No. 61/138,374, entitled “In-Line Coating of Yarn Prior to Creating a Fabric,” filed on Dec. 17, 2008, by the same inventor hereof, the disclosure of which is expressly incorporated herein by reference.

### BACKGROUND

1. Field of the Invention

The present invention relates to orthopedic implants. More particularly, the present invention relates to woven implants for cartilage replacement and to a method for making the same.

2. Description of the Related Art

Some implants for cartilage replacement are constructed of rigid materials, such as cobalt chromium. Although these implants may be strong enough for implantation into a load-bearing joint, such materials may cause opposing surfaces of the joint to wear.

Other implants for cartilage replacement are constructed of flexible materials, such as hydrogels. Although these implants provide smooth articular bearing surfaces, such materials may not withstand the loads of some joints, especially in the aqueous environment of the human body.

### SUMMARY

The present invention provides a woven implant for cartilage replacement having layered functionality. An exemplary woven implant may include a bottom layer, a top layer, and an intermediate layer. The bottom layer includes a plurality of interwoven fibers that are surface-treated to promote anchoring to bone. The top layer includes a plurality of interwoven fibers that are surface-treated to promote lubrication. The intermediate layer is located between the bottom layer and the top layer and includes a plurality of interwoven fibers that are surface-treated to promote soft tissue attachment. This exemplary woven implant may be strong enough for implantation into a load-bearing joint, while also having a smooth articular bearing surface.

According to an embodiment of the present invention, a method is provided for forming an orthopedic implant for cartilage replacement from a first plurality of fibers and a second plurality of fibers, each of the first and second plurality of fibers having a surface. The method includes the steps of: treating the surfaces of the first plurality of fibers to make the first plurality of fibers more hydrophilic than the second plurality of fibers; and after the treating step, weaving together the first plurality of fibers to form a top layer of the orthopedic implant and weaving together the second plurality of fibers to form a bottom layer of the orthopedic implant that is coupled to the top layer of the orthopedic implant, the top layer defining an articulating surface of the orthopedic implant and the bottom layer defining a bone-contacting surface of the orthopedic implant.

According to another embodiment of the present invention, a method is provided for forming an orthopedic implant for implantation into a cartilage defect site of a patient's body, the cartilage defect site being surrounded by remaining bone and remaining cartilage. The method includes the steps of: providing a first plurality of fibers and a second plurality of fibers, each of the first and second plurality of fibers having a surface; treating the surfaces of the first plurality of fibers to increase the hydrophilicity of the first plurality of fibers; after the treating step, weaving together the first plurality of fibers to form a top layer of the orthopedic implant and weaving together the second plurality of fibers to form a bottom layer of the orthopedic implant that is coupled to the top layer of the orthopedic implant, the orthopedic implant sized for implantation into the cartilage defect site with the bottom layer of the orthopedic implant positioned adjacent to the remaining bone and the top layer of the orthopedic implant positioned adjacent to the remaining cartilage.

According to yet another embodiment of the present invention, a woven orthopedic implant is provided for cartilage replacement having an articulating surface and a bone-contacting surface opposite the articulating surface. The orthopedic implant includes: a first plurality of fibers interwoven to form a top layer of the orthopedic implant, the top layer defining the articulating surface of the orthopedic implant, each of the first plurality of fibers having an exterior surface that is treated to promote articulation; a second plurality of fibers interwoven to form a bottom layer of the orthopedic implant, the bottom layer defining the bone-contacting surface of the orthopedic implant, each of the second plurality of fibers having an exterior surface that promotes bone attachment; and a third plurality of fibers interwoven to form an intermediate layer of the orthopedic implant coupled to both the top and bottom layers of the orthopedic implant, each of the third plurality of fibers having an exterior surface that promotes soft tissue attachment.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a cross-sectional view of an exemplary three-dimensional woven material;

FIG. 2 is a partial cross-sectional view of a knee joint, the knee joint including a femur, a tibia, and a patella, including an exemplary orthopedic prosthesis implanted into the femur;

FIG. 3 is a schematic representation of an exemplary method of the present invention; and

FIG. 4 is a graphical representation of the experimental results of fiber wettability tests.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

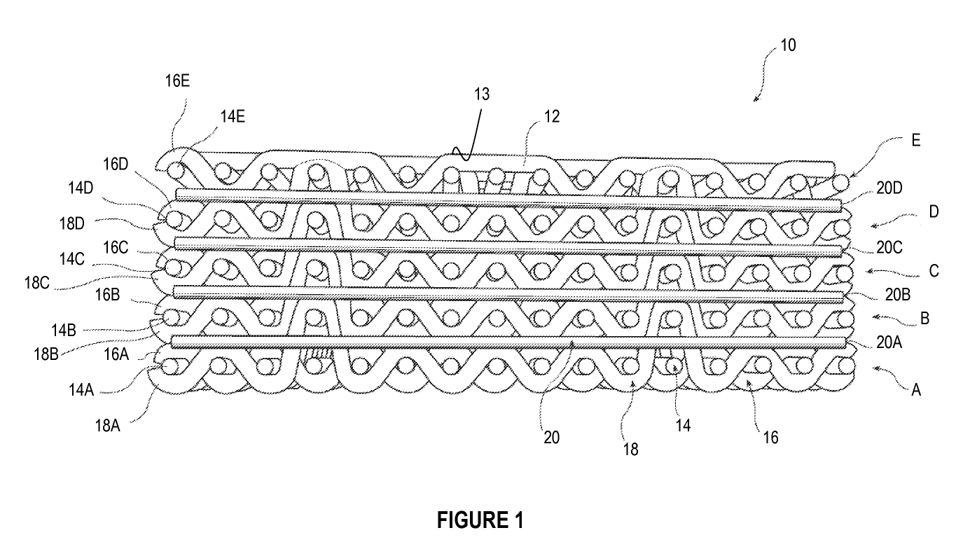

Referring to FIG. 1, an exemplary woven material is illustrated as three-dimensional woven material 10. Three-dimensional woven material 10 includes a plurality of interwoven, elongate fibers 12. Specifically, three-dimensional woven material 10 includes a plurality of weft fibers 14 (extending out of the page), a plurality of in-layer warp fibers 16, a plurality of out-of-layer warp fibers 18, and a plurality of between-layer warp fibers 20. Fibers 12 of three-dimensional woven material 10 may be made of various materials and may be provided in various diameters. Also, the particular weave pattern and weave density of three-dimensional woven material 10 may be varied. For example, three-dimensional woven material 10 may have a non-uniform porosity and strength to conform to the properties of natural human cartilage.

Each fiber 12, including each weft fiber 14, in-layer warp fiber 16, out-of-layer warp fiber 18, and between-layer warp fiber 20, may be made of one or more materials. For example, each fiber 12 may be a braided fiber made of multiple materials. Fibers 12 may be made of biocompatible materials including polymers (such as thermoplastics and hydrophilic hydrogels), acrylics, natural fibers, metals, glass fibers, carbon fibers, ceramics, or other suitable biocompatible materials. Exemplary polymers include propylene, polyester, high density polyethylene (HDPE), low density polyethylene (LDPE), ultra-high molecular weight polyethylene (UHMWPE), polycarbonate urethane, and polyetheretherketones (PEEK). Exemplary hydrophilic hydrogels include polyvinyl alcohol (PVA), polyvinylpyrrolidone (PVP), and polyethylene glycol (PEG). Exemplary acrylics include polymethyl methacrylate (PMMA). Exemplary natural fibers include elasin, keratin, silk, hydroxyl apatite (HA), collagen, and chitosan. Exemplary metals include stainless steel, titanium, titanium alloys, cobalt, nickel titanium alloy (nitinol), and tantalum. Exemplary ceramics include zirconia, alumina, and silica.

In the illustrated embodiment of FIG. 1, three-dimensional woven material 10 includes five layers A, B, C, D, E, of fibers 12. Specifically, layer A includes weft fibers 14A, in-layer warp fibers 16A, out-of-layer warp fibers 18A, and between-layer warp fibers 20A; layer B includes weft fibers 14B, in-layer warp fibers 16B, out-of-layer warp fibers 18B, and between-layer warp fibers 20B; layer C includes weft fibers 14C, in-layer warp fibers 16C, out-of-layer warp fibers 18C, and between-layer warp fibers 20C; layer D includes weft fibers 14D, in-layer warp fibers 16D, out-of-layer warp fibers 18D, and between-layer warp fibers 20D; and layer E includes weft fibers 14E and in-layer warp fibers 16E. Although five layers are shown, three-dimensional woven material 10 may include any number of layers.

Each layer A, B, C, D, E, is coupled to an adjacent layer through out-of-layer warp fibers 18. Specifically, out-of-layer warp fibers 18A couple layers A and B, out-of-layer warp fibers 18B couple layers B and C, out-of-layer warp fibers 18C couple layers C and D, and out-of-layer warp fibers 18D couple layers D and E. Although out-of-layer warp fibers 18 are shown joining together two adjacent layers, out-of-layer warp fibers 18 may couple together more than two layers. For example, out-of-layer warp fibers 18A could extend beyond layer B and into layer C, D, or E.

In an embodiment of the present invention, three-dimensional woven material 10 includes fibers 12 that form a generally rigid body. In another embodiment of the present invention, three-dimensional woven material 10 includes fibers 12 that form a generally flexible body. In yet another embodiment of the present invention, three-dimensional woven material 10 includes a stiffness gradient. Referring to the illustrated embodiment of FIG. 1, fibers 12 in layer A may be rigid, fibers 12 in layer E may be flexible, and fibers 12 in layers B, C, and D, may have stiffness characteristics between those of layers A and E. For example, layer A may include metallic fibers, layer B may include ceramic fibers, layer C may include thermoplastic fibers, layer D may include braided thermoplastic/hydrogel fibers, and layer E may include hydrogel fibers. Each out-of-layer warp fiber 18 may have a stiffness generally the same as its base layer or the layer it couples to its base layer. For example, out-of-layer warp fibers 18A of layer A may have a stiffness generally the same as fibers 12 of layer A or fibers 12 of layer B. Similarly, each between-layer warp fiber 20 may have a stiffness generally the same as either adjacent layer. For example, between-layer warp fibers 20A of layer A may have a stiffness generally the same as fibers 12 of layer A or fibers 12 of layer B.

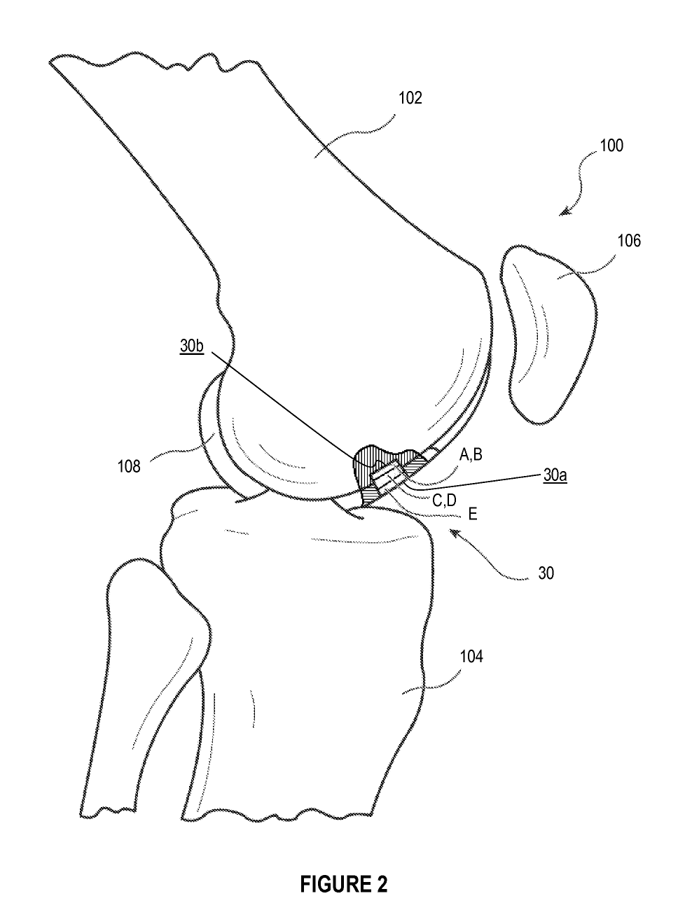

Referring next to FIG. 2, three-dimensional woven material 10 of FIG. 1 may form at least a portion of orthopedic implant 30. In the illustrated embodiment, implant 30 is implanted in knee joint 100, which includes femur 102, tibia 104, and patella 106. The portion of femur 102 that articulates with tibia 104 and patella 106 is surrounded by cartilage 108. Implant 30 is described and depicted as being implanted into femur 102 of knee joint 100. However, implant 30 may be implanted into other bones of the body, including, for example, tibia 104, a bone of the hip joint, a bone of the elbow joint, or a bone of the shoulder joint. According to an exemplary embodiment of the present invention, implant 30 may be used to repair and/or replace damaged cartilage 108.

Referring to FIGS. 1 and 2, individual fibers 12 of three-dimensional woven material 10 may be treated to alter the substantially cylindrical exterior surface 13 of each fiber 12. For example, individual fibers 12 of three-dimensional woven material 10 may be treated to alter the chemistry of exterior surface 13. Fibers 12 may be surface treated using various dry or wet treatments. Suitable dry treatments include corona or glow discharge treatments (such as atmospheric plasma treatments, flame plasma treatments, chemical plasma treatments, and gas plasma treatments), flame treatments, ozone treatments, ionized ray treatments (such as ultraviolet treatments and radiation treatments), electron beam treatments, and rough surface treatments. Suitable wet treatments include chemical agent treatments, polymer coatings, electrodepositing, and catalyst-aided grafting.

Gas plasma treatments, in particular, involve exciting a reactant gas to the plasma state of matter and introducing the excited gas to a substrate to fracture bonds along the surface of the substrate and initiate chemical reactions at the surface of the substrate. These broken bonds and chemical reactions may also occur at a limited depth beneath the surface of the substrate, but the bulk properties of the substrate generally are not altered.

According to an exemplary embodiment of the present invention, fibers 12 having surfaces 13 with various properties may be created, and these surface-treated fibers 12 may be layered to produce three-dimensional woven material 10 having a desired layered functionality. From this layered three-dimensional woven material 10 of FIG. 1, implant 30 of FIG. 2 having a desired layered functionality may be produced. For example, fibers 12 in layers A and B may be surface-treated to promote anchoring to surrounding bone, fibers 12 in layers C and D may be surface-treated to promote soft tissue ingrowth, and fibers 12 in layer E may be surface-treated to promote articulation and lubrication. As shown in FIG. 2, the upper-most fibers 12 in layer A define articulating surface 30a of implant 30, and the lower-most fibers 12 in layer E define bone-contacting surface 30b of implant 30. Each out-of-layer warp fiber 18 may undergo the same surface treatment as its base layer or the layer it couples to its base layer. For example, out-of-layer warp fibers 18B of layer B may undergo the same surface treatment as fibers 12 of layer B or fibers 12 of layer C. Similarly, each between-layer warp fiber 20 may undergo the same surface treatment as either adjacent layer. For example, between-layer warp fibers 20B of layer B may undergo the same surface treatment as fibers 12 of layer B or fibers 12 of layer C.

To promote anchoring to surrounding bone of femur 102, fibers 12 in layers A and B may be treated to become hydrophobic in nature. Hydrophobic fibers 12 may repel synovial fluid to permit bone growth into layers A and B of implant 30. Specifically, bone of femur 102 may grow into spaces between fibers 12 and into porous fibers 12 themselves. Alternatively, it has also been shown that hydrophilic materials may promote initial bone adherence, so it is within the scope of the present invention that some or all fibers 12 in layers A and B may be treated to become hydrophilic in nature.

To make fibers 12 hydrophobic in nature, fibers 12 may undergo gas plasma treatment with a fluorinated reactant gas, such as carbon tetrafluoride (CF₄), sulfur hexafluoride (SF₆), and perfluorohydrocarbons. When the fluorinated reactant gas is energized and exposed to fibers 12, hydrogen atoms along surface 13 of each treated fiber 12 may be substituted for fluorine atoms to create a non-polar, inert, Teflon-like surface 13. It is also within the scope of the present invention that fibers 12 may be sufficiently hydrophobic in nature as manufactured, without requiring subsequent surface treatments.

Also, to promote anchoring to surrounding bone of femur 102, fibers 12 in layers A and B may be roughened or etched to create binding sites for osteocytes and/or bio-active molecules. Such surface treatments may encourage a permanent attachment of implant 30 to femur 102.

In addition, to promote anchoring to surrounding bone of femur 102, fibers 12 in layers A and B may be manufactured or surface treated to include suitable proteins and/or peptides, such as arginine-glycine-aspartate (RGD) peptides, covalently bonded to surface 13 of each treated fiber 12. RGD peptides may be covalently bonded to fibers 12 via suitable functional groups, such as hydroxyl, amino, or carboxyl functional groups, on surface 13 of each treated fiber 12. Such functional groups may be introduced to fibers 12 by blending or co-polymerization. Also, such functional groups may be introduced to fibers 12 by chemical and physical treatments, similar to those treatments discussed above. For example, to deposit an amino functional group onto surfaces 13 of fibers 12, fibers 12 may undergo gas plasma treatment with ammonia as the reactant gas.

To promote soft tissue ingrowth, fibers 12 in layers C and D may be treated to become hydrophilic in nature. For example, polar functional groups, such as carboxyl functional groups or hydroxyl functional groups, may be deposited onto surface 13 of each treated fiber 12 using a gas plasma process. Hydrophilic fibers 12 may encourage soft tissue growth into layers C and D of implant 30. Specifically, soft tissue, such as cartilage 108, may grow into spaces between fibers 12 and into porous fibers 12 themselves. Such surface treatments may encourage a permanent attachment of implant 30 to cartilage 108 surrounding femur 102.

To promote low coefficient of friction articulation and lubrication, fibers 12 in surface layer E may be treated to encourage surface wetting. For example, polar functional groups, such as carboxyl functional groups or hydroxyl functional groups, may be deposited onto surface 13 of each treated fiber 12 using a gas plasma process. Also, fibers 12 in surface layer E may be treated to attract superficial zone proteins. It is within the scope of the present invention that fibers 12 in layer E may be treated using the same method as fibers 12 in layers C and D. It is also within the scope of the present invention that fibers 12 in layer E may be treated to become more hydrophilic than fibers 12 in layers C and D, and that fibers 12 in layers C and D may be treated to become more hydrophilic than fibers 12 in layers A and B. Such surface treatments may enhance articulation with adjacent structures of knee joint 100, including tibia 104 and patella 106, by binding superficial zone proteins common to native cartilage 108.

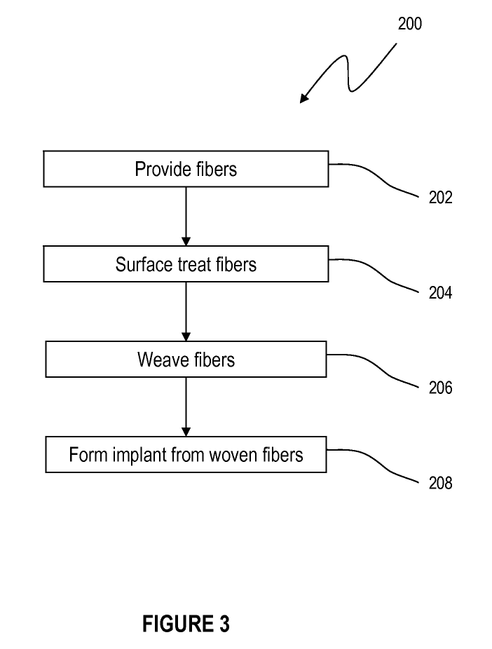

Referring next to FIG. 3, an exemplary method 200 is provided to manufacture implant 30 (FIG. 2). Beginning with step 202, biocompatible fibers 12 (FIG. 1) are provided having desired physical properties. As discussed above, exemplary fibers 12 include, for example, ultra-high molecular weight polyethylene (UHMWPE) fibers. One known process for manufacturing fibers is described in U.S. Pat. No. 4,415,521 to Mininni et al., the disclosure of which is incorporated herein by reference. Exemplary fibers, including Dyneema Purity™ SGX fibers, are currently generally available from DSM Biomedical of the Netherlands. Dyneema Purity™ SGX fibers, in particular, are non-degradable, UHMWPE fibers having a high tensile strength (e.g. average tenacity at break of 32 cN/dtex), a lower profile than steel or polyester fibers of the same strength, and a smooth exterior (e.g. coefficient of friction of less than 0.10).

Continuing to step 204 of FIG. 3, surfaces 13 of fibers 12 (FIG. 1) are treated. As mentioned above, fibers 12 may be surface treated using various dry or wet treatments. Suitable dry treatments include corona or glow discharge treatments (such as atmospheric plasma treatments, flame plasma treatments, chemical plasma treatments, and gas plasma treatments), flame treatments, ozone treatments, ionized ray treatments (such as ultraviolet treatments and radiation treatments), electron beam treatments, and rough surface treatments. Suitable wet treatments include chemical agent treatments, polymer coatings, electrodepositing, and catalyst-aided grafting. One known method for surface treating fibers is described in U.S. Pat. No. 3,853,657 to Lawton, the disclosure of which is incorporated herein by reference.

Following step 204, fibers 12 are woven together in step 206 in the desired order and density to form three-dimensional woven material 10. As discussed above, fibers 12 in layers A and B may be surface-treated to promote anchoring to surrounding bone, fibers 12 in layers C and D may be surface-treated to promote soft tissue ingrowth, and fibers 12 in layer E may be surface-treated to promote articulation and lubrication. The fibers may be woven together using known weaving processes, such as the process described in U.S. Pat. No. 4,154,267 to Orr et al., the disclosure of which is incorporated herein by reference. Also, the fibers may be woven together according to processes currently performed by Secant Medical, LLC of Perkasie, Pa.

Advantageously, weaving in step 206 after surface treating in step 204 produces an implant that may have more than two functional layers, including functional top, bottom, and intermediate layers. Also, the implant maintains its desired bulk properties. Surface treating the final bulk implant after weaving, on the other hand, produces at most a functional top layer and a functional bottom layer. Also, depending on the treatment method, surface treating the final bulk implant after weaving may impact only the top-most and bottom-most fibers, not intermediate fibers.

Continuing to step 208 of FIG. 3, three-dimensional woven material 10 (FIG. 1) is processed into implant 30 (FIG. 2) for implantation into the body. For example, three-dimensional woven material 10 may be formed into the desired shape and size, cleaned, sterilized, and packaged, prior to implantation.

### Example

### Wettability Testing

Fibers were subjected to various gas plasma treatments to evaluate the impact of such treatments on fiber wettability. The fibers included strands of 220 dtex Dyneema Purity™ SGX yarn, available from DSM Biomedical of the Netherlands. The following treatments were performed using a gas plasma device supplied by PVA TePla America, Inc. of Corona, California: (1) addition of hydroxyl functional group; (2) fluorination; (3) oxidation; and (4) addition of carboxyl functional group.

Each of the four treated yarns and a fifth untreated yarn was cut into five pieces of equal lengths. Individually, one end of each piece of yarn was tied to a ring stand while the other end of the yarn was allowed to hang and contact 40 mL of room temperature Crystal Violet solution, available from Becton, Dickinson and Company of Franklin Lakes, N.J.

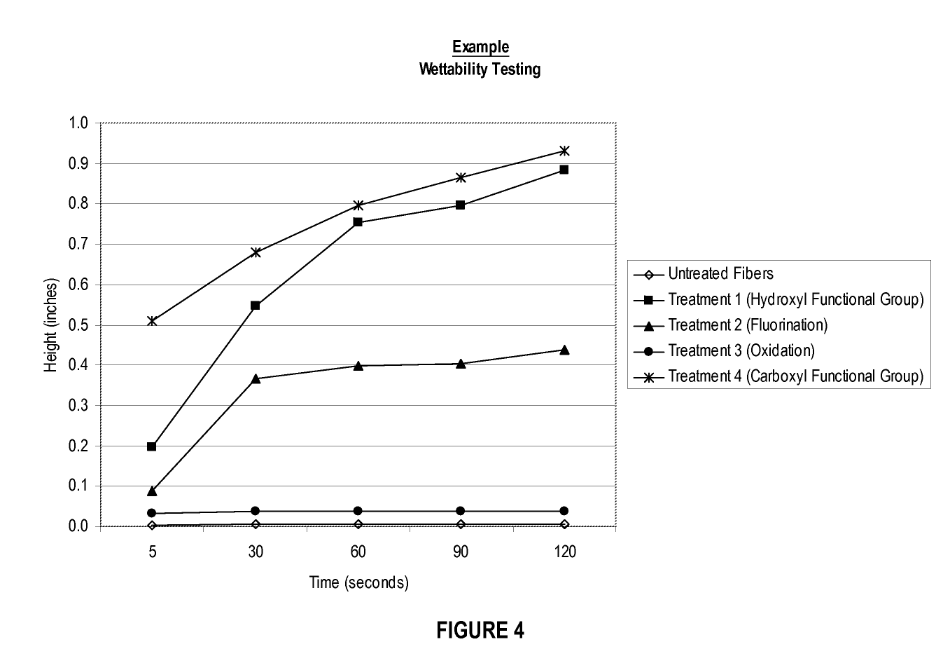

Over time, the fibers absorbed the solution. The height or distance (in inches) that the colored solution visibly climbed into the fiber was measured at the following time increments: 5 seconds, 30 seconds, 60 seconds, 90 seconds, and 120 seconds. The graphical results of this experiment are set forth in FIG. 4. The most hydrophilic fibers were those with carboxyl functional groups and hydroxyl functional groups added to the surface.

While this invention has been described as having preferred designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of forming an orthopedic implant for cartilage replacement from a first plurality of fibers and a second plurality of fibers, each of the first and second plurality of fibers having a surface, the method comprising the steps of:
treating the surfaces of the first plurality of fibers to make the first plurality of fibers more hydrophilic than the second plurality of fibers; and
after the treating step, weaving together the first plurality of fibers to form a top layer of the orthopedic implant and weaving together the second plurality of fibers to form a bottom layer of the orthopedic implant that is coupled to the top layer of the orthopedic implant, the top layer defining an articulating surface of the orthopedic implant and the bottom layer defining a bone-contacting surface of the orthopedic implant.

2. The method of claim 1, wherein the treating step comprises adding one of a hydroxyl functional group and a carboxyl functional group to the surfaces of the first plurality of fibers.

3. The method of claim 1, wherein the treating step comprises increasing the polarity of the surfaces of the first plurality of fibers.

4. The method of claim 1, further comprising the step of treating the surfaces of the second plurality of fibers before the weaving step to alter the surfaces of the second plurality of fibers.

5. The method of claim 4, wherein treating the surfaces of the second plurality of fibers comprises increasing the hydrophobicity of the second plurality of fibers.

6. The method of claim 4, wherein treating the surfaces of the second plurality of fibers comprises roughening the surfaces of the second plurality of fibers.

7. The method of claim 4, wherein treating the surfaces of the second plurality of fibers comprises bonding one of a protein and a peptide to the surfaces of the second plurality of fibers.

8. The method of claim 7, wherein the peptide comprises arginine-glycine-aspartate.

9. The method of claim 1, further comprising the steps of:
providing a third plurality of fibers; and
weaving together the third plurality of fibers to form an intermediate layer of the orthopedic implant located between the top and bottom layers, the third plurality of fibers being more hydrophobic than the first plurality of fibers and more hydrophilic than the second plurality of fibers.

10. The method of claim 1, wherein both the first and second plurality of fibers comprise ultra-high molecular weight polyethylene.

11. A method of forming an orthopedic implant for implantation into a cartilage defect site of a patient's body, the cartilage defect site being surrounded by remaining bone and remaining cartilage, the method comprising the steps of:
providing a first plurality of fibers and a second plurality of fibers, each of the first and second plurality of fibers having a surface;
treating the surfaces of the first plurality of fibers to increase the hydrophilicity of the first plurality of fibers;
after the treating step, weaving together the first plurality of fibers to form a top layer of the orthopedic implant and weaving together the second plurality of fibers to form a bottom layer of the orthopedic implant that is coupled to the top layer of the orthopedic implant, the orthopedic implant sized for implantation into the cartilage defect site with the bottom layer of the orthopedic implant positioned adjacent to the remaining bone and the top layer of the orthopedic implant positioned adjacent to the remaining cartilage.

12. The method of claim 11, wherein the treating step comprises adding one of a hydroxyl functional group and a carboxyl functional group to the surfaces of the first plurality of fibers.

13. The method of claim 11, further comprising the step of treating the surfaces of the second plurality of fibers before the weaving step to alter the surfaces of the second plurality of fibers, the treated surfaces of the second plurality of fibers differing from the treated surfaces of the first plurality of fibers.

14. The method of claim 13, wherein the step of treating the surfaces of the second plurality of fibers comprises making the surfaces of the second plurality of fibers more hydrophobic in nature.

15. The method of claim 13, wherein the step of treating the surfaces of the second plurality of fibers comprises roughening the surfaces of the second plurality of fibers.

16. The method of claim 13, wherein the step of treating the surfaces of the second plurality of fibers comprises bonding one of a protein and a peptide to the surfaces of the second plurality of fibers.

17. The method of claim 11, further comprising the steps of:
providing a third plurality of fibers; and
weaving together the third plurality of fibers to form an intermediate layer of the orthopedic implant located between the top and bottom layers, the third plurality of fibers being more hydrophobic than the first plurality of fibers and more hydrophilic than the second plurality of fibers.

18. A woven orthopedic implant for cartilage replacement having an articulating surface and a bone-contacting surface opposite the articulating surface, the orthopedic implant comprising:
a first plurality of fibers interwoven to form a top layer of the orthopedic implant, the top layer defining the articulating surface of the orthopedic implant, each of the first plurality of fibers having an exterior surface that is treated to promote articulation;
a second plurality of fibers interwoven to form a bottom layer of the orthopedic implant, the bottom layer defining the bone-contacting surface of the orthopedic implant, each of the second plurality of fibers having an exterior surface that promotes bone attachment; and
a third plurality of fibers interwoven to form an intermediate layer of the orthopedic implant coupled to both the top and bottom layers of the orthopedic implant, each of the third plurality of fibers having an exterior surface that promotes soft tissue attachment.

19. The orthopedic implant of claim 18, wherein the treated exterior surfaces of the first plurality of fibers are more hydrophilic than exterior surfaces of the second plurality of fibers.

20. The orthopedic implant of claim 18, wherein the treated exterior surfaces of the first plurality of fibers include one of a hydroxyl functional group and a carboxyl functional group bonded to the treated exterior surfaces.

21. The orthopedic implant of claim 18, wherein the second plurality of fibers are more rigid than the first plurality of fibers.

22. The orthopedic implant of claim 18, wherein both the first and second plurality of fibers comprise ultra-high molecular weight polyethylene.
